(19) United States
(12) Patent Application Publication
(10) Pub. No.: US 2015/0216603 A1
(43) Pub. Date: Aug. 6, 2015

(54) NONWOVEN FABRIC AND POLYURETHANE COMPOSITE MATERIALS AND METHODS FOR PRODUCING THE SAME

(71) Applicant: 3M INNOVATIVE PROPERTIES COMPANY, SAINT PAUL, MN (US)

(72) Inventors: Jing Ma, Shanghai (CN); Lei Sun, Shanghai (CN); Dunshen Zhu, Shanghai (CN); Jing Huang, Shanghai (CN)

(21) Appl. No.: 14/423,798

(22) PCT Filed: Aug. 23, 2013
(86) PCT No.: PCT/US2013/056312

§ 371 Date: Feb. 25, 2015

(30) Foreign Application Priority Data
Aug. 29, 2012 (CN) 201210313679.0

(51) Int. Cl. A61B 19/08; B32B 27/12; B32B 38/00; B32B 37/24; B32B 37/06; B32B 5/02; B32B 3/26

(52) U.S. Cl. CPC A61B 19/088; B32B 5/022; B32B 27/12; B32B 3/26; B32B 37/24; B32B 37/06; B32B 38/0036; B32B 2262/0292; B32B 2307/7265; B32B 2535/00; B32B 2037/243; B32B 2375/00; B32B 2371/00

(57) ABSTRACT

The disclosure provides a composite material of nonwoven fabric and polyurethane produced without use of an adhesive and a method for producing the same. The composite material comprises a nonwoven fabric and a nonporous or porous polyurethane film, wherein the polyurethane film is laminated on the nonwoven fabric. The disclosure provides a method for producing the composite material as described above, comprising the steps of: firstly casting a polyurethane solution on a substrate so as to form a film, then laminating the film and a nonwoven fabric by flexibly adjusting the lamination process so as to form a composite material comprising the nonporous or porous polyurethane film and the nonwoven fabric. The composite material comprising a nonporous polyurethane film and a nonwoven fabric can be used for medical articles such as surgical gowns and drapes and the like, and the composite material comprising a porous polyurethane film and a nonwoven fabric can be used for medical articles such as absorbent pads, adhesive bandages, wound dressings and the like.

### TECHNICAL FIELD

The disclosure relates to a medical composite material, and in particularly, to a composite material for disposable surgical gowns, disposable surgical drapes and nonwoven fabric absorbent pads and the like.

### BACKGROUND ART

Disposable surgical gowns and disposable surgical drapes are generally required to have superior waterproof and ethanol-proof and bacteria resistant properties. A nonwoven fabric has been generally laminated with a polyethylene film to impart it with the waterproof and bacteria resistant properties and the laminate has been widely used as medical materials, such as protective clothing, surgical gowns, and surgical drapes and the like. However, the nonwoven fabric laminated with the polyethylene has disadvantages of hard feeling, low moisture vapor permeability and poor air permeability, which is uncomfortable for medical care personnel to wear due to mugginess, itching or the like and affects the working efficiency.

PCT/EP2008/050232 has disclosed a laminated material comprising the components bonded together: (i) a thermoplastic polyurethane-based film, wherein the thermoplastic polyurethane is based on a polyether glycol which is produced by alkylating a starting bifunctional material and in which the ethylene oxide is used as the alkylene oxide and has a weight fraction of at least 20 wt % based on the total weight of the alkylene oxide used; and (ii) a thermoplastic polyurethane-based nonwoven fiber material.

W02007/114295A1 has disclosed a sheet substrate obtained by laminating a polyurethane nonwoven fabric and a polyurethane film. The above polyurethane nonwoven fabric is a nonwoven fabric formed by stacking fibers with an average fiber diameter of 50 μm or less in a continuous and substantially unconverged manner and jointing the fibers themselves, and the above polyurethane film has micropores of 10 μm or less on both sides of the film and interconnected micropores in the thickness direction of the film.

CN201109221Y and US2009/0081911A1 have disclosed a composite nonwoven fabric with resistance to bacterial and waterproof properties, which is formed by laminating three layers of materials where the surface layer is a water absorbable layer fabricated from a water absorbable nonwoven fabric, the intermediate layer is a waterproof layer fabricated from a polyethylene film, a polyurethane film or waterproof and air permeable film and the inner layer is an resistance to bacterial layer fabricated from a bacterial-resistible nonwoven fabric.

CN2791093 has disclosed a composite fabric which is formed by thermally pressing three layers of nonwoven fabrics where the top layer is a spun-laced fabric or hot through-air fabric, the bottom layer is a polypropylene coating or a spunbonded fabric, and the intermediate layer is a polyurethane air permeable fabric with micropores.

CN2629475 has disclosed a medical protective clothing formed by laminating a nonwoven fabric layer treated via a coating technology and a microporous film with a binder, wherein the nonwoven fabric layer can be obtained by using a spun-laced nonwoven fabric of polyester filaments or a polypropylene (meltblown) nonwoven fabric and treating it via a coating technology with a flame retardant, an antistatic agent and a water repelling agent, and the microporous film uses a hydrophilic polyurethane film (TPU) which can prevent the pass-through of viruses and bacteria.

However, the nonwoven fabric composite materials disclosed in the above prior art have disadvantages of hard feeling, poor comfortability, bad moisture permeability and the like to some different extents.

### SUMMARY OF THE DISCLOSURE

In order to overcome the disadvantages of hard feeling, poor comfortability, bad moisture vapor permeability and the like of the existing medical nonwoven fabric composite materials, the disclosure provides a novel medical nonwoven fabric composite material and a method for producing the same.

In an aspect of the disclosure, a medical nonwoven fabric composite material is provided. The medical nonwoven fabric composite material comprises a nonwoven fabric and a polyurethane film, wherein the polyurethane film is laminated on the nonwoven fabric.

In one embodiment, the polyurethane film is a nonporous polyurethane film.

In one embodiment, the polyurethane film is a porous polyurethane film.

In another aspect of the disclosure, a method for producing the medical nonwoven fabric composite material comprising a nonwoven fabric and a nonporous polyurethane film, comprising the steps of:

1) casting a first polyurethane solution formed from a first polyurethane resin and a first solvent on a substrate to form a first polyurethane film on the substrate, wherein the first polyurethane film has a solvent content of 30 to 45% by weight;

2) laminating the first polyurethane film and a nonwoven fabric under a condition of 0.3 to 0.9 MPa to form a composite film; and

3) heating the composite film under the condition of 105 to 125° C. to form the composite material consisting of the nonwoven fabric and the polyurethane film.

In another aspect of the disclosure, a method for producing the medical nonwoven fabric composite material comprising a nonwoven fabric and a nonporous polyurethane film, comprising the steps of:

1) casting a first polyurethane solution comprising a first polyurethane resin and a first solvent on a first substrate to from a first polyurethane film on the first substrate, wherein the first polyurethane film has a solvent content of 30 to 45% by weight;

2) laminating the first polyurethane film and a nonwoven fabric under a condition of 0.3 to 0.9 MPa to form a first composite film;

3) heating the first composite film under the condition of 105 to 125° C. to form the first composite material comprising the nonwoven fabric and the first polyurethane film;

4) casting a second polyurethane solution comprising a second polyurethane resin and a second solvent on a second substrate to from a second polyurethane film on the second substrate, wherein the second polyurethane film has a solvent content of 30 to 45% by weight;

5) laminating the second polyurethane film and the first composite material under a condition of 0.3 to 0.9 MPa to form a second composite film; and

6) heating the second composite film under the condition of 105 to 125° C. to form the second composite material consisting of the second polyurethane film and the first composite material;

wherein the steps 4) to 6) are performed for one or more times to produce a composite material comprising two or more polyurethane films and the nonwoven fabric.

In a still another aspect of the disclosure, a method for producing the medical nonwoven fabric composite material comprising a nonwoven fabric and a porous polyurethane film, comprising the steps of:

1) casting a first polyurethane solution comprising a first polyurethane resin and a first solvent on a substrate to form a first polyurethane film on the substrate, wherein the first polyurethane film has a solvent content of 65 to 75 by weight;

2) laminating the first polyurethane film and a nonwoven fabric under a condition of 0.5 to 1.0 MPa to form a composite film; and

3) heating the composite film under the condition of 105 to 125° C. to form the composite material comprising the nonwoven fabric and the polyurethane film.

In another aspect of the disclosure, a method for producing the medical nonwoven fabric composite material comprising a nonwoven fabric and a porous polyurethane film, comprising the steps of:

1) casting a first polyurethane solution comprising a first polyurethane resin and a first solvent on a first substrate to form a first polyurethane film on the substrate, wherein the first polyurethane film has a solvent content of 65 to 75 by weight;

2) laminating the first polyurethane film and a nonwoven fabric under a condition of 0.5 to 1.0 MPa to form a first composite film; and

3) heating the first composite film under the condition of 105 to 125° C. to form the first composite material comprising the nonwoven fabric and the first polyurethane film;

4) casting a second polyurethane solution formed from a second polyurethane resin and a second solvent on a second substrate to form a second polyurethane film on the second substrate, wherein the second polyurethane film has a solvent content of 60 to 80 by weight;

5) laminating the second polyurethane film and the first composite material under a condition of 0.5 to 1.0 MPa to form a second composite film; and

6) heating the second composite film under the condition of 105 to 125° C. to form the second composite material comprising the second polyurethane film and the first composite material;

wherein the steps 4) to 6) are performed for one or more times to produce a composite material comprising two or more polyurethane films and the nonwoven fabric.

By laminating a specific polymer material selected and used with a nonwoven fabric while adjusting the lamination process, the disclosure can produce a novel medical nonwoven fabric composite material having a certain structure. The novel medical nonwoven fabric composite material provided by the disclosure possesses advantages of superior waterproof and ethanol-proof and bacteria resistant properties, as well as good moisture vapor and air permeability and comfortable feeling and the like, and can be used for protective clothing, surgical gowns, surgical drapes, absorbent pads and the like.

### DESCRIPTION OF THE DRAWINGS

FIG. 1 is a side sectional schematic diagram of the composite material of a single nonporous polyurethane film and a nonwoven fabric according to one embodiment of the disclosure.

FIG. 2 is a side sectional schematic diagram of the composite material of a plurality of nonporous polyurethane films and a nonwoven fabric according to one embodiment of the disclosure.

FIG. 3 is a side sectional schematic diagram of the composite material of a single porous polyurethane film and a nonwoven fabric according to one embodiment of the disclosure.

FIG. 4 is a side sectional schematic diagram of the composite material of a plurality of porous polyurethane films and a nonwoven fabric according to one embodiment of the disclosure.

### DETAILED DESCRIPTION

The embodiments of the disclosure are described in detail below and are illustrated in the drawings where like or similar references represent like or similar elements. The embodiments described with reference to the drawings below are illustrative with the aim of explaining the disclosure and cannot be construed to limit the disclosure.

### Medical Nonwoven Fabric Composite Material

“Porous polyurethane film” means a polyurethane film, which can be passed through by liquid water from either side of the polyurethane film.

“Nonporous polyurethane film” means a polyurethane film, which cannot be passed through by liquid water from either side of the polyurethane film.

The medical nonwoven fabric composite material provided by the disclosure comprises a nonwoven fabric and a polyurethane film, wherein the polyurethane film is directly laminated on the nonwoven fabric without use of an adhesive.

The nonwoven fabric may be made of one or more materials of: polyethylene terephthalate, polypropylene, polyphenylene sulfide, polyterephthalamide and polyisophthalamide, and preferably one or more materials of polyethylene terephthalate and polypropylene.

The polyurethane in the polyurethane film may be one or more selected from the group consisting of an aliphatic polyether polyurethane, an aliphatic polyester polyurethane, an aromatic polyether polyurethane, and an aromatic polyester polyurethane, and preferably an aromatic polyether polyurethane. The aromatic polyether polyurethane may be one or more selected from the group consisting of a solvent type polyurethane, an aqueous polyurethane, a thermoplastic polyurethane, and a thermosetting polyurethane, and preferably a solvent type aromatic polyether polyurethane. The solvent type aromatic polyether polyurethane comprises the one or more isocyanates of: toluene diisocyanate (TDI), diphenylmethane diisocyanate (MDI), and p-phenylene diisocyanate (PPDI), and preferably a solvent type aromatic polyether polyurethane comprising diphenylmethane diisocyanate (MDI).

As the polyurethane film, a nonporous polyurethane film may be selected and used, or a porous polyurethane film may be selected and used.

The nonporous polyurethane film or porous polyurethane film may be separately produced by the respective methods described in the portion “Method for producing a medical nonwoven fabric composite material” of this specification.

The medical nonwoven fabric composite material provided by the disclosure may comprise one nonporous polyurethane film and a layer of nonwoven fabric.

In the medical nonwoven fabric composite material comprising one nonporous polyurethane film and a nonwoven fabric, the nonwoven fabric has a thickness of 0.06 to 0.6 mm, and preferably 0.1 to 0.5 mm; and the nonporous polyurethane films have a thickness of 5 to 50 μm, and preferably 8 to 30 μm. By selecting the nonporous polyurethane film having a thickness within this range, the good bonding power can be provided between the polyurethane film and the nonwoven fabric and the resulting medical nonwoven fabric composite material can be provided with superior waterproof, ethanol-proof, bacteria resistant and moisture permeable properties.

The medical nonwoven fabric composite material comprising one nonporous polyurethane film and a nonwoven fabric can be further used for producing medical materials such as surgical gowns, surgical drapes, protective clothing, disinfecting wrap cloths, masks and the like. The surgical gowns made of this medical nonwoven fabric composite material have features of comfortability and fast perspiration, and the surgical drapes made of this medical nonwoven fabric composite material have markedly reduced surface resistance and can markedly reduce the potential risk of generating static electricity during the operation.

The medical nonwoven fabric composite material provided by the disclosure may comprise a plurality of nonporous polyurethane films and a layer of nonwoven fabric.

In the medical nonwoven fabric composite material comprising a plurality of nonporous polyurethane films and a nonwoven fabric, the nonwoven fabric has a thickness of 0.06 to 0.6 mm, and preferably 0.1 to 0.5 mm; and the first nonporous polyurethane film adjacent to the nonwoven fabric has a thickness of 10 to 50 μm, and preferably 12 to 30 μm, and each of the other polyurethane films has a thickness of 8 to 20 μm. Preferably, the thickness of the first polyurethane film laminated to the nonwoven fabric is greater than that of each of the other polyurethane films, the first thicker polyurethane film may be bonded well with the nonwoven fabric, and the second thinner polyurethane film may apply the medical nonwoven fabric composite material with good moisture vapor permeability waterproof, ethanol-proof and bacteria resistance, wherein the moisture vapor transmission rate (MVTR) may reach to 3800 g/m²/24h, which is 40 times greater than that of the material of polyethylene film laminated with nonwoven fabric.

The medical nonwoven fabric composite material comprising a plurality of nonporous polyurethane films and a nonwoven fabric may be further used for producing medical materials such as absorbent pads, adhesive bandages, wound dressings and the like.

The medical nonwoven fabric composite material provided by the disclosure may comprise a single porous polyurethane film and a single of nonwoven fabric.

The porous polyurethane film has a thickness of 10 to 50 μm, and preferably 15 to 30 μm, an open pore size of 0.05 to 0.8 mm and preferably 0.2 to 0.5 mm, and an open pore density of 1000 to 3000 pores/inch.

The nonwoven fabric has a thickness of 0.4 to 1.5 mm, and preferably 0.6 to 1.2 mm.

The medical nonwoven fabric composite material comprising a single porous polyurethane film and a nonwoven fabric has better biocompatibility, softness and comfortability and can be adjusted in terms of the rate of absorbing penetrative liquids by the nonwoven fabric through the open pore size of the porous polyurethane film. Therefore, this medical nonwoven fabric composite material comprising a single porous polyurethane film and a nonwoven fabric may be further used for producing medical materials such as absorbent pads, wound dressings and the like so as to maintain the dryness and comfort of the wound and reduce the propagation of bacteria.

The medical nonwoven fabric composite material provided by the disclosure may comprise a plurality of porous polyurethane films and a single nonwoven fabric.

In the plurality of porous polyurethane films, a first polyurethane film laminated to the nonwoven fabric has a thickness ranging from 10 to 50 μm, and each of the other polyurethane films has a thickness ranging from 8 to 20 μm; and preferably, the thickness of the first polyurethane film adjacent to the nonwoven fabric is greater than that of each of the other polyurethane films.

The nonwoven fabric has a thickness of 0.4 to 1.5 mm, and preferably 0.6 to 1.2 mm.

The medical nonwoven fabric composite material comprising a plurality of porous polyurethane films and a nonwoven fabric has better biocompatibility, softness and comfortability, and may be further used for producing medical materials such as absorbent pads, adhesive bandages, wound dressings and the like.

### Method for Producing the Medical Nonwoven Fabric Composite Material

“Laminating”, as used herein, means applying two materials to each other under a certain pressure, and in the disclosure, applying a nonwoven fabric material to a substrate coated with a polyurethane film under a certain pressure (for example, 0.5 to 1.0 MPa).

“Casting”, as used herein, means a process of coating a liquid-state compound on a substrate and then forming a film by drying.

In the disclosure, percents, parts or ratios of the components are based on the weight, unless otherwise noted.

The method for producing the medical nonwoven fabric composite material provided in the disclosure comprises the steps of:

1) casting a polyurethane solution comprising a polyurethane resin and a solvent so as to form a polyurethane film having a solvent content of 30 to 75% by weight based on 100% by weight of the polyurethane film;

2) laminating the polyurethane film and a nonwoven fabric under a condition of 0.3 to 1.0 MPa to form a composite film; and

3) heating the composite film under the condition of 105 to 125° C. to form the medical nonwoven fabric composite material.

The method for producing the nonwoven fabric is as follows: subjecting the fibers to air-laid or mechanical carding to form a web, then consolidated it by spun-lacing, needling, chemical bonding or hot melt adhering (including hot rolling and hot through-air bonding), and finally forming a nonwoven fabric by a post finishing process.

In the disclosure, the nonwoven fabric is not particularly limited, so long as the nonwoven fabric has a substantially even thickness, surfaces free of impurities and relatively great holes. The nonwoven fabric may be made of at least one material selected from the group consisting of polyethylene terephthalate, polypropylene, polyphenylene sulfide, polyterephthalamide and polyisophthalamide and the like, and preferably polyethylene terephthalate and polypropylene. The thickness of the nonwoven fabric may range from 0.06 to 1.5 mm, and preferably 0.1 to 1.2 mm. The non-limiting examples of the nonwoven fabric comprise a spunbond nonwoven fabric provided by Wenzhou Changlong Textile Technology Company Limited, a spun-laced nonwoven fabric provided by DuPont-Dayuan Non-Woven Fabric Co., Ltd of Dalian Ruiguang Nonwoven Group, a hot through-air nonwoven fabric provided by Guangzhou ES Fiber Co., Ltd., and a needled nonwoven fabric provided by Changshu Lixin Nonwoven Fabric Co., Ltd.

The polyurethane solution may be cast on a substrate so as to form a polyurethane film.

The substrate may be, but not limited to, a paper-based substrate, a film-based substrate, an aluminum foil-based substrate, a polyolefin-based substrate, a nonwoven fabric-based substrate, and preferably a paper-based substrate or a film-based substrate. The paper-based substrate or film-based substrate comprises, but is not limited to, a release paper or a release film with a coating. The release paper or release film has a coating layer with low surface energy on at least one surface, wherein the coating layer comprises one or more compounds of: a silicone, a fluoride, a fluorosilicone copolymer, and a compound with a long chain polyolefin as the side chain. The release paper or release film, and preferably the release paper or release film with a basis weight of 64 to 120 g/m² can be commercially available from Shanghai Paoyan Industrial Technology Co., Ltd and Guangzhou Loparex Paper Products Co, Ltd.

The medical nonwoven fabric composite material comprising one or more nonporous or porous polyurethane film and a nonwoven fabric may be produced by directly coating a film with the polyurethane solution and directly laminating the polyurethane film and the nonwoven fabric according to the method provided by the disclosure without the use of an adhesive. The method enables a flexible process, a convenient operation and a simple desired apparatuses.

### The Production of the Medical Nonwoven Fabric Composite Material Comprising a Nonporous Polyurethane Film and a Nonwoven Fabric

Specifically, the disclosure provides a method for producing the medical nonwoven fabric composite material comprising one nonporous polyurethane film and a nonwoven fabric, comprising the steps of:

Step 1a: a polyurethane solution is obtained by mixing a suitable polyurethane resin selected and used with a suitable solvent; wherein the polyurethane solution has a solvent content of 60 to 80% by weight and preferably 65 to 75% by weight and a viscosity of 3000 to 15000 cp and preferably 3000 to 8000 cp; the polyurethane resin is preferably an aromatic polyether polyurethane resin, for example, MVT75-AT3 provided by Lubrizol Company, Estane 58245 polyurethane resin provided by Lubrizol Company, V-5854 polyurethane resin provided by Vix. Co. Ltd; and as the solvent, one or more of N,N′-dimethylformamide (DMF) and butanone (MEK), and preferably the solvent consisting of N,N′-dimethylformamide and butanone may be selected and used, and the solvent with a volume ratio of N,N′-dimethylformamide to butanone of 3:7 to 7:3 and in particularly 6:4 is particularly preferable.

Step 2a: the polyurethane solution obtained in the step 1a is cast on a substrate (release paper) so as to form a polyurethane film, the polyurethane film has a solvent content of 30 to 45% by weight based on 100% by weight of the polyurethane film; the polyurethane film has a thickness of 8 to 30 μm after being dried; and the cast process may be carried out by devices such as manual coating machine (comma roller) or other suitable known coating apparatuses or the like.

Step 3a: the polyurethane film obtained in the step 2a is laminated with the nonwoven fabric so as to form a composite film; the nonwoven fabric has a thickness of 0.1 to 0.5 mm; and the laminating process may be carried out by devices such as manual bonding roller or other suitable known kneading apparatuses or the like at the condition of 0.3 to 0.9 MPa.

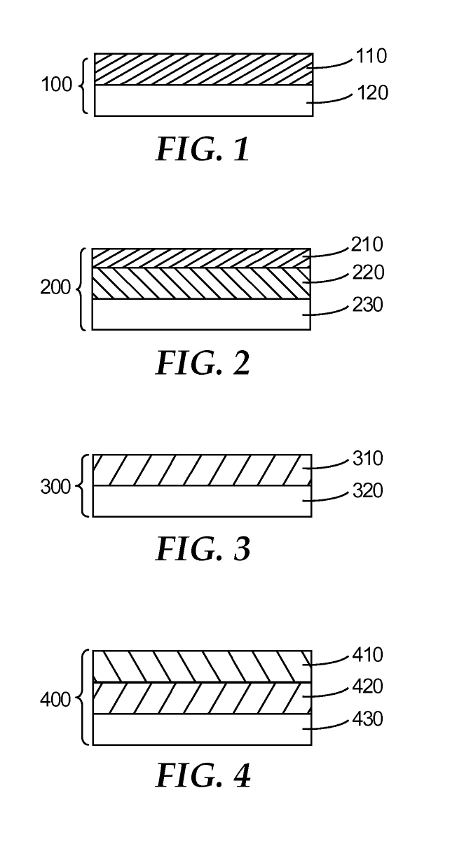

Step 4a: the composite film obtained in the step 3a is placed and dried in the oven at 105 to 125° C., and after peeling off the substrate (release paper), the medical nonwoven fabric composite material comprising a single nonporous polyurethane film and a nonwoven fabric as shown in FIG. 1 can be obtained.

Specifically, the disclosure provides a method for producing the medical nonwoven fabric composite material consisting of two nonporous polyurethane films and a nonwoven fabric, comprising the steps of:

Step 1b: a polyurethane solution is obtained by mixing a suitable polyurethane resin selected and used with a suitable solvent; wherein the polyurethane solution has a solvent content of 60 to 80% by weight and preferably 65 to 75% by weight and a viscosity of 3000 to 15000 cp and preferably 3000 to 8000 cp; the polyurethane resin is preferably an aromatic polyether polyurethane resin, for example, MVT75-AT3 provided by Lubrizol Company, Estane 58245 polyurethane resin provided by Lubrizol Company, V-5854 polyurethane resin provided by Vix. Co. Ltd; and as the solvent, one or more of N,N′-dimethylformamide (DMF) and butanone (MEK), and preferably the solvent consisting of N,N′-dimethylformamide and butanone may be selected and used, and the solvent with a volume ratio of N,N′-dimethylformamide to butanone of 3:7 to 7:3 and in particularly 6:4 is particularly preferable.

Step 2b: the polyurethane solution obtained in the step 1b is cast on a substrate (release paper) so as to form a polyurethane film, the polyurethane film has a solvent content of 30 to 45% by weight based on 100% by weight of the polyurethane film; the polyurethane film has a thickness of 8 to 20 μm after being dried; and the cast process may be carried out by devices such as manual coating machine (comma roller) or other suitable known coating apparatuses or the like.

Step 3b: the polyurethane film obtained in the step 2b is laminated with the medical nonwoven fabric composite material comprising a single nonporous polyurethane film and a nonwoven fabric produced in the step 4a so as to form a composite film; and the laminating process may be carried out by devices such as manual bonding roller or other suitable known kneading apparatuses or the like at the condition of 0.3 to 0.9 MPa.

Step 4b: the composite film obtained in the step 3b is placed and dried in the oven at 105 to 125° C., and after peeling off the substrate (release paper), the medical nonwoven fabric composite material comprising two nonporous polyurethane films and a nonwoven fabric as shown in FIG. 2 can be obtained.

If it is required to produce the medical nonwoven fabric composite material comprising three nonporous polyurethane films and a nonwoven fabric, the polyurethane film obtained in the step 2b is laminated with the medical nonwoven fabric composite material comprising two nonporous polyurethane films and a nonwoven fabric produced in the step 4a by devices such as manual bonding roller or other suitable known kneading apparatuses or the like at the condition of 0.3 to 0.9 MPa so as to form a composite film, and the resulting composite film is then placed and dried in the oven at 105 to 125° C., and after peeling off the substrate (release paper), the medical nonwoven fabric composite material comprising three nonporous polyurethane films and a nonwoven fabric can be obtained.

According to the above method, the medical nonwoven fabric composite material comprising four or more nonporous polyurethane films and a nonwoven fabric can be also obtained.

### The Production of the Medical Nonwoven Fabric Composite Material Comprising a Porous Polyurethane Film and a Nonwoven Fabric

Specifically, the disclosure provides a method for producing the medical nonwoven fabric composite material comprising one porous polyurethane film and a nonwoven fabric, comprising the steps of:

Step 1c: a polyurethane solution is obtained by mixing a suitable polyurethane resin selected and used with a suitable solvent; wherein the polyurethane solution has a solvent content of 55 to 80% by weight and preferably 65 to 75% by weight and a viscosity of 3000 to 15000 cp and preferably 3000 to 8000 cp; the polyurethane resin is preferably an aromatic polyether polyurethane resin, for example, MVT75-AT3 provided by Lubrizol Company, Estane 58245 polyurethane resin provided by Lubrizol Company, V-5854 polyurethane resin provided by Vix. Co. Ltd; and as the solvent, one or more of N,N′-dimethylformamide (DMF) and butanone (MEK), and preferably the solvent consisting of N,N′-dimethylformamide and butanone may be selected and used, and the solvent with a volume ratio of N,N′-dimethylformamide to butanone of 3:7 to 7:3 and in particularly 6:4 is particularly preferable.

Step 2c: the polyurethane solution obtained in the step 1c is cast on a release paper so as to form a polyurethane film, wherein the polyurethane film has a solvent content of 55 to 80% by weight, preferably 65 to 75% by weight based on 100% by weight of the polyurethane film; the polyurethane film has a thickness of 15 to 30 μm after being dried; and the cast process may be carried out by devices such as manual coating machine (comma roller) or other suitable known coating apparatuses or the like.

Step 3c: the polyurethane film obtained in the step 2c is laminated with the nonwoven fabric so as to form a composite film; wherein the nonwoven fabric has a thickness of 0.6 to 1.2 mm; and the laminating process may be carried out by devices such as manual bonding roller or other suitable known kneading apparatuses or the like. A porous polyurethane composite film having an open pore size diameter of 0.2 to 0.5 mm and an open pore density of 1000 to 3000 pores/inch can be flexibly produced depending on the magnitude of laminating pressure.

Step 4c: the composite film obtained in the step 3c is placed and dried in the oven at 105 to 125° C., and after peeling off the substrate (release paper), the medical nonwoven fabric composite material consisting of a single nonporous polyurethane film and a nonwoven fabric as shown in FIG. 3 can be obtained.

Specifically, the disclosure provides a method for producing the medical nonwoven fabric composite material comprising two porous polyurethane films and a nonwoven fabric, comprising the steps of:

Step 1d: a polyurethane solution is obtained by mixing a suitable polyurethane resin selected and used with a suitable solvent; wherein the polyurethane solution has a solvent content of 55 to 80% by weight and preferably 65 to 75% by weight and a viscosity of 3000 to 15000 cp and preferably 3000 to 8000 cp; the polyurethane resin is preferably an aromatic polyether polyurethane resin, for example, MVT75-AT3 provided by Lubrizol Company, Estane 58245 polyurethane resin provided by Lubrizol Company, V-5854 polyurethane resin provided by Vix. Co. Ltd; and as the solvent, one or more of N,N′-dimethylformamide (DMF) and butanone (MEK), and preferably the solvent consisting of N,N′-dimethylformamide and butanone may be selected and used, and the solvent with a volume ratio of N,N′-dimethylformamide to butanone of 3:7 to 7:3 and in particularly 6:4 is particularly preferable.

Step 2d: the polyurethane solution obtained in the step 1d is cast on a substrate (release paper) so as to form a polyurethane film, wherein the polyurethane film has a solvent content of 55 to 80% by weight, preferably 65 to 75% by weight based on 100% by weight of the polyurethane film; the polyurethane film has a thickness of 15 to 30 μm after being dried; and the cast process may be carried out by devices such as manual coating machine (comma roller) or other suitable known coating apparatuses or the like.

Step 3d: the polyurethane film obtained in the step 2d is laminated with the nonwoven fabric so as to form a composite film; the nonwoven fabric has a thickness of 0.6 to 1.2 mm; and the laminating process may be carried out by devices such as manual bonding roller or other suitable known kneading apparatuses or the like at the condition of 0.5 to 1.0 MPa. A porous polyurethane composite film having an opening diameter of 0.2 to 0.5 mm and an open pore density of 1000 to 3000 pores/inch can be flexibly produced depending on the magnitude of laminating pressure.

Step 4d: the composite film obtained in the step 3d is placed and dried in the oven at 105 to 125° C., and after peeling off substrate (release paper), the medical nonwoven fabric composite material comprising two nonporous polyurethane films and a nonwoven fabric as shown in FIG. 4 can be obtained.

If it is required to produce the medical nonwoven fabric composite material comprising three nonporous polyurethane films and a nonwoven fabric, the polyurethane film obtained in the step 2d is laminated with the medical nonwoven fabric composite material comprising two nonporous polyurethane films and a nonwoven fabric produced in the step 4d by devices such as manual bonding roller, manual blade or other suitable known kneading apparatuses or the like at the condition of 0.5 to 1.0 MPa so as to form a composite film, and the resulting composite film is then placed and dried in the oven at 105 to 125° C., and after peeling off the release paper, the medical nonwoven fabric composite material comprising three nonporous polyurethane films and a nonwoven fabric can be obtained.

According to the above method, the medical nonwoven fabric composite material comprising four or more porous polyurethane films and a nonwoven fabric can be also obtained.

FIG. 1 is a side sectional schematic diagram of the composite material of a single nonporous polyurethane film and a nonwoven fabric according to one embodiment of the disclosure, wherein the composite material 100 of a single nonporous polyurethane film and a nonwoven fabric comprises a single nonporous polyurethane film 110 and a nonwoven fabric 120. FIG. 2 is a side sectional schematic diagram of the composite material of a plurality of nonporous polyurethane films and a nonwoven fabric according to one embodiment of the disclosure, wherein the composite material 200 of a plurality of nonporous polyurethane films and a nonwoven fabric comprises a nonwoven fabric 230, a thicker nonporous polyurethane film 220 and a thinner nonporous polyurethane film 210. FIG. 3 is a side sectional schematic diagram of the composite material of a porous polyurethane film and a nonwoven fabric according to one embodiment of the disclosure, wherein the composite material 300 of a porous polyurethane film and a nonwoven fabric comprises a porous polyurethane film 310 and a nonwoven fabric 320. FIG. 4 is a side sectional schematic diagram of the composite material of a plurality of porous polyurethane films and a nonwoven fabric according to one embodiment of the disclosure, wherein the composite material 400 of a plurality of porous polyurethane and a nonwoven fabric comprises porous polyurethane films 410, 420, and a nonwoven fabric 430.

### EXAMPLES

The disclosure is described in more detail by ways of examples below. It would note that these examples are illustrative of the disclosure and do not limit the disclosure in any way. The percents, parts and ratios and the like used in the disclosure are based on the weight and the temperature used means Celsius degree, unless specifically noted.

The methods for measuring the properties of the medical nonwoven fabric composite material provided by the disclosure are described below so as to evaluate the properties such as waterproofness, ethanol-proofness, resistance to bacteria, moisture permeability, surface resistance and the like of the medical nonwoven fabric composite material, respectively.

### Waterproofness and Ethanol-Proofness

The waterproofness and ethanol-proofness of the samples were tested using ionized water and ethanol added with a dye, and the wetting process of water drop or ethanol on the samples per unit time was observed. If it is impossible for water and ethanol to wet the sample within 10 min, the sample can be evaluated as having superior waterproofness and ethanol-proofness. If it is impossible for water and ethanol to wet the sample within 5 min, the sample can be evaluated as having good waterproofness and ethanol-proofness.

### Resistance to Bacteria

The resistance to bacteria of the samples was tested according to YY/T0471.5-2004 standard. The specific operation steps and the instruments and materials used were as follows:

1. Instruments and Materials

1. 1 Replicate Organism Detection and Counting Plate (RODAC)

1.2 100 g of weight

1.3 Nutrient broth

1.4 Nutrient agar culture medium

1.5 Serratia marcescens 8100 culture

2. Steps

2.1 The serratia marcescens was cultured with the nutrient broth at 20 to 25° C. for 24 h so as to obtain a bacterial content of about 109/mL;

2.2 The RODAC plate was filled fully with the nutrient agar culture medium;

2.3 The bacterial solution to be tested was soaked with a sterile metal ring, and a “X” shape was inoculated on the surface of the RODAC plate, with the length of each cross line being no more than 2 cm;

2.4 The culture plate was cultured at 20 to 25° C. for 24 h so as to grow the bacterial colonies;

2.5 A sterile sample (with a surface area of at least of 5 cm×5 cm) was placed on the RODAC plate with a sterile operation so as to cover the “X” shaped bacterial culture;

2.6 The RODAC plate which was filled fully with flesh blood agar culture medium and which was not inoculated with bacteria was placed on the samples, and a 100 g of weight was then added on the RODAC plate so as to generate continuous pressure on the material;

2.7 The entire culture plate was cultured at 20 to 25° C. for 24 h;

2.8 The top layer of blood agar RODAC plate was removed, covered, and further cultured at 20 to 25° C. for 24 h;

2.9 Whether there were growing serratia marcescens at the surface where the culture plate was covered by the sample was observed (note: a red colony of serratia marcescens occurred on the agar); and

2. 10 The steps were repeated for further two samples.

3. Results

If there was growing serratia marcescens for one or more of the three RODAC plates, the sample failed to pass the test.

### Moisture Vapor Transmission Rate (MVTR)

The Moisture vapor transmission rate (MVTR) of the sample was tested under the condition of 40° C. and 20% relative humidity according to ASTM-E96M-05 standard. If MVTR is greater than 3000 g/m² 0.24h, it was proved that the sample had superior moisture permeability. The specific steps and conditions were as follows:

1. About 50 ml of water was charged into a glass bottle;

2. The sample was applied to the adhesive side of the aluminum foil ring, and it would note that the sample would be placed in the center of elliptic hole of the aluminum foil ring;

3. In order to ensure the alignment with the second aluminum foil ring, the first aluminum foil ring (with adhesive surface upward) was placed on a plane. The samples were aligned with the center and applied at the first aluminum foil. Then, the second aluminum foil ring (with adhesive surface downward) was placed on a sample such that the elliptic holes of the two aluminum rings are coincided with each other. The aluminum foil/sample/aluminum foil was pressurized and flatten with a finger. Note that there is no wrinkle or hole;

4. A rubber gasket was placed on the mouth of the bottle, and the aluminum foil/sample and aluminum foil were then placed on the gasket. If there was a adhesive coating on the sample, the adhesive side was placed downward (if a surface of the sample was a film, and the other surface is clothing or nonwoven fabric, the surface having the film was placed downward);

5. The lid of the bottle was lightly screwed on, and the bottle was placed on a metal holder and put into an aging box. The aging box was set at the condition of 40° C.±1° C. and a relative humidity of 20%±2% for 4 h;

6. The lid of the bottle was screwed on tightly in the aging box (the forefinger was placed on the lid of the bottle and the sample such that the sample and the lid of the bottle were at the same level so as to avoid the generation of expansion) and a rubber gasket was placed in position;

7. The sample was taken out from the aging box and immediately weighted using an analytical balance (an initial weight of W1) with a precision of 0.01 g;

8. The bottle was placed back into the aging box and kept for at least for 18 h;

9. The sample was taken out from the aging box and immediately weighted using an analytical balance (a final weight of W2) with a precision of 0.01 g; and

10. The moisture permeability was calculated (unit: g/m²/24h).

MVTR=(W1−W2)/S/T 24 h

wherein,

W1=the initial weight (g) W2=the final weight (g)

S=an testing area of the sample (m²)

T=testing time (h), wherein the testing time should be greater than 18h.

### Surface Resistance

A surface resistance of the sample was tested with a surface resistance tester TREK-152P-CE (manufacturer: Trek, Inc). The surface resistance tester has a measuring range of 10⁴ to 10¹³ ohms and a measuring precision of ±5%. A surface resistance value, a resistivity and a volume resistance value of the sample was tested with the surface resistance tester according to the measuring technology complying with the criterion of the ANSI/ESD Association.

### Materials Used in the Examples:

| Name | Property | Source |
|---|---|---|
| spunbonded and | blue, basis weight of 65 g, | commercially available from 3M China |
| polyethylene-laminated | thickness of 0.25 mm | Limited |
| nonwoven fabric(surgical | | |
| drape) | | |
| polypropylene spunbonded | white, basis weight of 30 g, | commercially available from Wenzhou |
| nonwoven fabric | thickness of 0.13 mm | Changlong Textile Technology Company |
| | | Limited |
| polypropylene spun-laced | white, basis weight of 30 g, | co mmercially available from Dalian |
| nonwoven fabric | thickness of 0.2 mm | Ruiguang Nonwoven Group Co. Ltd. |
| polypropylene hot | white, basis weight of 150 g, | commercially available from Guangzhou |
| through-air nonwoven fabric | thickness of 0.5 mm | ES Fiber Co., Ltd. |
| Polypropylene needled | white, basis weight of 185 g, | commercially available from Changshu |
| nonwoven fabric | thickness of 1.03 mm | Lixin Nonwoven Fabric Co., Ltd. |
| aromatic polyethe | a molecular weight greater | commercially available from Lubrizol or |
| | than 100,000 | |
| release paper 64 | basis weight of 64 g, single | commercially available from Shanghai |
| release paper 80 | basis weight of 80 g, single | commercially available from Guangzhou |
| | sided white glassine | |
| release paper 120 | basis weight of 120 g, single | commercially available from Guangzhou |
| manual bonding roller | gas pressure range of 0.1 to | commercially available from 3M China |

### Comparative Example 1

The spunbonded and polyethylene-laminated nonwoven fabric with a basis weight of 65 g, and thickness of 0.25 mm from 3M was selected and used.

### Example 1

Preparing the medical nonwoven fabric composite material comprising a single nonporous polyurethane film and polypropylene spunbonded nonwoven fabrics, comprising the specific steps as follows:

Step 1: Dilution of Polyurethane Solution

The aromatic polyether polyurethane resin V-5854 (provided by Vix. Co., Ltd.) and a mixed solvent of N,N′-dimethylformamide (DMF) and butanone (MEK) (a volume ratio of N,N′-dimethylformamide to butanone of 6:4) were mixed so as to produce a polyurethane solution with a solvent content of 75% by weight and a viscosity of 4000 cp;

Step 2: Casting

The polyurethane solution obtained in the step 1 was cast on the release paper 64 with a manual coater (comma roller) so as to form a polyurethane film, the polyurethane film had a solvent content of 40% by weight based on 100% by weight of the polyurethane film and had a thickness of 20 μm after being dried.

Step 3: Laminating

The polyurethane film obtained in the step 2 was laminated with the polypropylene spunbonded nonwoven fabric having a basis weight of 30 g at the condition of 0.5 MPa with a manual bonding roller so as to form a composite film, wherein the polypropylene spunbonded nonwoven fabric had a thickness of 0.25 mm.

Step 4: Drying

The composite film obtained in the step 3 was placed and dried in the oven at 110° C. and after peeling off the release paper, the medical nonwoven fabric composite material comprising a single nonporous polyurethane film and a polypropylene spunbonded nonwoven fabric as shown in FIG. 1 was obtained.

### Example 2

Preparing the medical nonwoven fabric composite material comprising two nonporous polyurethane films and a polypropylene spunbonded nonwoven fabric, comprising the specific steps as follows:

The steps 1 to 4 were the same as those of the example 1.

Step 5: Further Casting

The polyurethane solution obtained in the step 1 was cast on the release paper 80 with a manual coater (comma roller) so as to form a polyurethane film, the polyurethane film had a solvent content of 45% by weight based on 100% by weight of the polyurethane film and had a thickness of 10 μm after being dried.

Step 6: Further Lamination

The polyurethane film obtained in the step 5 was further laminated with a medical nonwoven fabric composite material comprising a nonporous polyurethane film and a polypropylene spunbonded nonwoven fabric produced in the example 1 at the condition of 0.3 MPa with a manual bonding roller so as to form a composite film.

Step 7: Further Drying

The composite film obtained in the step 6 was placed in the oven at 120° C. for 5 min, and after peeling off the release paper, the medical nonwoven fabric composite material comprising two nonporous polyurethane films and a polypropylene spunbonded nonwoven fabric as shown in FIG. 2 was obtained.

### Example 3

Preparing the Medical Nonwoven Fabric Composite Material Comprising Two Nonporous polyurethane films and a spun-laced nonwoven fabric, comprising the specific steps as follows:

The operation steps in the example 2 were repeated by using the spun-laced nonwoven fabric having a basis weight of 40 g so as to produce the medical nonwoven fabric composite material comprising two nonporous polyurethane films and a spun-laced nonwoven fabric, wherein the first polyurethane film adjacent to the spun-laced nonwoven fabric had a thickness of 20 μm and the second polyurethane film had a thickness of 10 μm.

### Example 4

Preparing the medical nonwoven fabric composite material comprising two nonporous polyurethane films and a hot through-air nonwoven fabric, comprising the specific steps as follows:

The operation steps in the example 2 were repeated by using the spun-laced nonwoven fabric having a basis weight of 15 g so as to produce the medical nonwoven fabric composite material comprising two nonporous polyurethane films and a hot through-air nonwoven fabric, wherein the first polyurethane film adjacent to the spun-laced nonwoven fabric had a thickness of 20 μm and a second polyurethane film had a thickness of 10 μm.

### Example 5

Preparing the medical nonwoven fabric composite material comprising two nonporous polyurethane films and a polypropylene spunbonded nonwoven fabric. The specific steps were the same as those of the example 2, except that the first polyurethane film adjacent to the polypropylene spunbonded nonwoven fabric had a thickness of 30 μm.

### Example 6

Preparing the medical nonwoven fabric composite material comprising two nonporous polyurethane films and a polypropylene spunbonded nonwoven fabric. The specific steps were the same as those of the example 2, except that the second polyurethane film which was not adjacent to the polypropylene spunbonded nonwoven fabric had a thickness of 20 μm.

### Example 7

Preparing the medical nonwoven fabric composite material comprising a single porous polyurethane film and a spun-laced nonwoven fabric, comprising the specific steps as follows:

Step 1: Dilution of Polyurethane Solution

The aromatic polyether polyurethane resin MVT75-AT3 (provided by Lubrizol company) and a mixed solvent of N,N′-dimethylformamide (DMF) and butanone (MEK) (a volume ratio of N,N′-dimethylformamide to butanone of 6:4) were mixed so as to produce a polyurethane solution with a solvent content of 65% by weight and a viscosity of 8000 cp;

Step 2: Casting

The polyurethane solution obtained in the step 1 was cast on a release paper 120 with a manual coater (comma roller) so as to form a polyurethane film such that the polyurethane film had a solvent content of 65% by weight based on 100% by weight of the polyurethane film and had a thickness of 25 μm after being dried.

Step 3: Lamination

The polyurethane film obtained in the step 2 was laminated with the spun-laced nonwoven fabric having a basis weight of 185 g at the condition of 0.6 MPa with a manual bonding roller so as to form a composite film, wherein the spun-laced nonwoven fabric had a thickness of 0.6 mm.

Step 4: Drying

The composite film obtained in the step 3 was placed and dried in the oven at 115° C., and after peeling off the release paper, the medical nonwoven fabric composite material comprising a single porous polyurethane film and a spun-laced nonwoven fabric as shown in FIG. 3 was obtained, wherein the porous polyurethane film had a number average size of open pores of 0.2 mm and an open pore density of 2800 pores/inch.

### Example 8

Preparing the medical nonwoven fabric composite material comprising a single porous polyurethane film and a spun-laced nonwoven fabric was produced by the wet application. The specific steps were the same as those of the example 7, except that the lamination pressure was 1.0 MPa, and the porous polyurethane film had a number average size of open pores of 0.5 mm and an open pore density of 1700 pores/inch².

### Example 9

Preparing the medical nonwoven fabric composite material comprising two porous polyurethane film and a spun-laced nonwoven fabric, comprising the specific steps as follows:

The steps 1 to 4 were the same as those of the example 7.

Step 5: Further Casting

The polyurethane solution obtained in the step 1 was cast on the release paper 80 with a manual coater (comma roller) so as to form a polyurethane film, the polyurethane film had a solvent content of 65% by weight based on 100% by weight of the polyurethane film and had a thickness of 25 μm after being dried.

Step 6: Further Lamination

The polyurethane film obtained in the step 5 was further laminated with a medical nonwoven fabric composite material comprising a nonporous polyurethane film and a spun-laced nonwoven fabric produced in the example 7 at the condition of 0.7 MPa with a manual bonding roller so as to form a composite film.

Step 7: Further Drying

The composite film obtained in the step 6 was placed in the oven at 120° C. for 5 min, and after peeling off the release paper, the medical nonwoven fabric composite material comprising two nonporous polyurethane films and a polypropylene spunbonded nonwoven fabric as shown in FIG. 4 was obtained.

The properties of the medical nonwoven fabric composite materials produced according to the comparative example 1 and the examples 1 to 6 are shown in the Table 1.

TABLE 1

| | waterproofness | | Moisture vapor | |
|---|---|---|---|---|
| | and | resistance | transmission | surface |
| | ethanol- | to | rate (MVTR) | resistance |
| | proofness | bacteria | g/m²/24 h | Ω(×10 to 12) |
| Example 1 | good | superior | 4087 | 0.07 |
| Example 2 | superior | superior | 3929 | 0.04 |
| Example 3 | superior | superior | 3904 | 0.01 |
| Example 4 | superior | superior | 3802 | 0.03 |
| Example 5 | superior | superior | 3345 | 0.03 |
| Example 6 | superior | superior | 3374 | 0.03 |
| Comparative | superior | superior | 96 | 2.61 |
| Example 1 | | | | |

Further, as known from the Examples 7 and 8, taking the spun-laced nonwoven fabric as an example, the open pore size of the polyurethane film can be conveniently adjusted by adjusting the lamination pressure.

According to Table 1, as compared with the material laminated with a polyethylene film on a surface of a nonwoven fabric, the composite material comprising a nonporous polyurethane film and a nonwoven fabric can possess not only superior waterproof and ethanol-proof and bacteria resistant properties, but also excellent moisture permeability and anti-static properties, which is 34 to 40 times greater than that of the polyethylene film and spunbonded nonwoven fabric of the comparative example. Therefore, the composite material comprising a nonporous polyurethane film and a spun-laced nonwoven fabric can be further used for producing medical materials such as surgical gowns, surgical drapes, protective clothing, disinfecting wrap cloths, masks and the like.

According to Example 7, 8 or 9, the composite material comprising a porous polyurethane film and a nonwoven fabric can possess advantages of good moisture and air permeability and comfortable feeling and the like; Further, taking the spun-laced nonwoven fabric as an example, the open pore size of the polyurethane film in the composite material can be conveniently adjusted by adjusting the lamination pressure. Therefore, the composite material comprising a porous polyurethane film and a spun-laced nonwoven fabric can be further used for producing medical articles such as absorbent pads, adhesive bandages, and wound dressings and the like and is suitable for the long-term use for the patients.

1. A composite material comprising one or more polyurethane films and a nonwoven fabric, wherein the polyurethane films are directly laminated on the nonwoven fabric without use of an adhesive.

2. The composite material according to claim 1, wherein the polyurethane film is an aromatic polyether polyurethane film.

3. The composite material according to claim 1, wherein the polyurethane film is a nonporous polyurethane film or a porous polyurethane film.

4. The composite material according to claim 3, wherein the polyurethane film is a nonporous polyurethane film and the nonporous polyurethane film has a thickness of 5 to 50 μm.

5. (canceled)

6. The composite material according to claim 3, wherein the polyurethane film is a porous polyurethane film and the porous polyurethane film has a thickness of 10 to 50 μm.

7. The composite material according to claim 6, wherein the porous polyurethane film has an open pore size of 0.05 to 0.8 mm and an open pore density of 1000 to 3000 pores/inch².

8. The composite material according to claim 1, wherein the composite material comprising a plurality of polyurethane films and a nonwoven fabric, wherein a first polyurethane film adjacent to the nonwoven fabric has a thickness of 10 to 50 μm, each of the other polyurethane films has a thickness of 8 to 20 μm, and the thickness of the first polyurethane film adjacent to the nonwoven fabric is greater than that of each of the other polyurethane films.

9. (canceled)

10. The composite material according to claim 1, wherein the nonwoven fabric is made of at least one material selected from the group consisting of polyethylene terephthalate and polypropylene.

11. The composite material according to claim 3, wherein the polyurethane film is a nonporous polyurethane film, and the nonwoven fabric has a thickness of 0.06 to 0.6 mm.

12. The composite material according to claim 3, wherein the polyurethane film is a porous polyurethane film, and the nonwoven fabric has a thickness of 0.4 to 1.5 mm.

13. A method for producing the composite material according to claim 1, comprising the steps of:
1) casting a first polyurethane solution comprising a first polyurethane resin and a first solvent on a substrate to form a first polyurethane film on the substrate, wherein the first polyurethane film has a solvent content of 30 to 45% by weight;
2) laminating the first polyurethane film and a nonwoven fabric under a condition of 0.3 to 0.9 MPa to form a composite film; and
3) heating the composite film under the condition of 105 to 125° C. to form the composite material comprising the nonwoven fabric and the polyurethane film.

14. A method for producing the composite material according to claim 1, comprising the steps of:
1) casting a first polyurethane solution comprising a first polyurethane resin and a first solvent on a first substrate to form a first polyurethane film on the first substrate, wherein the first polyurethane film has a solvent content of 30 to 45% by weight;
2) laminating the first polyurethane film and a nonwoven fabric under a condition of 0.3 to 0.9 MPa to form a first composite film;
3) heating the first composite film under the condition of 105 to 125° C. to form the first composite material comprising the nonwoven fabric and the first polyurethane film;
4) casting a second polyurethane solution comprising a second polyurethane resin and a second solvent on a second substrate to form a second polyurethane film on the second substrate, wherein the second polyurethane film has a solvent content of 30 to 45% by weight;
5) laminating the second polyurethane film and the first composite material under a condition of 0.3 to 0.9 MPa to form a second composite film; and
6) heating the second composite film under the condition of 105 to 125° C. to form the second composite material comprising the second polyurethane film and the first composite material;
wherein the steps 4) to 6) are performed for one or more times to produce a composite material comprising two or more polyurethane films and the nonwoven fabric.

15. The method according to claim 13, wherein the first or second polyurethane resin is an aromatic polyether polyurethane.

16. The method according to claim 13, wherein the first or second polyurethane solution has a solvent content of 55 to 80% by weight and a viscosity of 3000 to 15000 cp.

17. (canceled)

18. (canceled)

19. The method according to claim 13, wherein the first or second solvent consists of N,N′-dimethylformamide and butanone, wherein a volume ratio of N,N′-dimethylformamide to butanone is from 3:7 to 7:3.

20. A method for producing the composite material according to claim 1, comprising the steps of:
1) casting a first polyurethane solution comprising a first polyurethane resin and a first solvent on a substrate to form a first polyurethane film on the substrate, wherein the first polyurethane film has a solvent content of 60 to 80% by weight;
2) laminating the first polyurethane film and a nonwoven fabric under a condition of 0.5 to 1.0 MPa to form a composite film; and
3) heating the composite film under the condition of 105 to 125° C. to form the composite material comprising the nonwoven fabric and the polyurethane film.

21. A method for producing the composite material according to claim 1, comprising the steps of:
1) casting a first polyurethane solution comprising a first polyurethane resin and a first solvent on a first substrate to form a first polyurethane film on the substrate, wherein the first polyurethane film has a solvent content of 60 to 80% by weight;
2) laminating the first polyurethane film and a nonwoven fabric under a condition of 0.5 to 1.0 MPa to form a first composite film; and
3) heating the first composite film under the condition of 105 to 125° C. to form the first composite material comprising the nonwoven fabric and the first polyurethane film;
4) casting a second polyurethane solution comprising a second polyurethane resin and a second solvent on a second substrate to form a second polyurethane film on the second substrate by volatilizing a portion of the second solvent, wherein the second polyurethane film has a solvent content of 60 to 80% by weight;
5) laminating the second polyurethane film and the first composite material under a condition of 0.5 to 1.0 MPa to form a second composite film; and
6) heating the second composite film under the condition of 105 to 125° C. to form the second composite material comprising the second polyurethane film and the first composite material;
wherein the steps 4) to 6) are performed for one or more times to produce a composite material comprising two or more polyurethane films and the nonwoven fabric.

22. The method according to claim 20, wherein the first or second polyurethane resin is an aromatic polyether polyurethane.

23. The method according to claim 20, wherein the first or second polyurethane solution has a solvent content of 55 to 80% by weight and a viscosity of 3000 to 15000 cp.

24. (canceled)

25. (canceled)

26. The method according to claim 20, wherein the first or second solvent consists of N,N′-dimethylformamide and butanone, wherein a volume ratio of N,N-dimethylformamide to butanone is from 3:7 to 7:3.
